(19) United States
(12) Patent Application Publication
(10) Pub. No.: US 2022/0049043 A1
(43) Pub. Date: Feb. 17, 2022

(54) Use of Biomass to Produce Polyoxymethylene Copolymers

(71) Applicant: Celanese International Corporation, Irving, TX (US)

(72) Inventors: Klaus Kurz, Kelsterbach (DE); Kirsten Markgraf, Weinheim (DE); Michael Hoffmockel, Niedernhausen (DE); Kai-Uwe Tönnes, Kongernheim (DE)

(21) Appl. No.: 17/401,541

(22) Filed: Aug. 13, 2021

Related U.S. Application Data
(60) Provisional application No. 63/065,767, filed on Aug. 14, 2020.

(51) Int. Cl. C08G 4/00; C07D 323/06; C08L 59/04; C08G 2/08

(52) U.S. Cl. CPC C08G 4/00; C08G 2/08; C08L 59/04; C07D 323/06

(57) ABSTRACT

The present disclosure is directed to a process for producing a polyoxymethylene polymer or paraformaldehyde in an environmentally friendly and sustainable manner. The polyoxymethylene polymer or paraformaldehyde can be produced so as to be carbon neutral or even carbon negative. In one aspect, the polyoxymethylene polymer or paraformaldehyde is formed from a biogas or from a recycled gas. The biogas and the recycled gas are used to produce the components needed to form the polymer.

### RELATED APPLICATIONS

The present application is based upon and claims priority to U.S. Provisional Patent Application Ser. No. 63/065,767, having a filing date of Aug. 14, 2020, which is incorporated herein by reference.

### BACKGROUND

Polyacetal polymers, which are commonly referred to as polyoxymethylene polymers, have become established as exceptionally useful engineering materials in a variety of applications. For instance, because polyoxymethylene polymers have excellent mechanical properties, fatigue resistance, abrasion resistance, chemical resistance, and moldability, they are widely used in constructing polymer articles, such as articles for use in the automotive industry, the electrical industry, consumer appliance industry, the medical industry, and the like.

Polyoxymethylene polymers are capable of being compounded with various different additives for changing and/or improving various properties. For instance, polyoxymethylene polymers can be combined with tribological modifiers for applications where low friction surfaces are desired. In other embodiments, reinforcing fillers and fibers can be incorporated into the polymer for increasing strength and toughness properties. In another aspect, impact modifiers can be added to the polymer to increase impact resistance.

Polyoxymethylene polymers are typically produced from formaldehyde monomers that are derived from fossil-based products. The production of polyoxymethylene polymers can also produce carbon emissions. Recently, however, many companies large and small have pledged to be carbon neutral within a particular period of time. Microsoft, for instance, has committed to be carbon negative by 2030. Amazon, on the other hand, has pledged to be carbon neutral by 2040. Unilever, which manufactures tens of thousands of consumer goods, has also pledged to be carbon neutral by 2039.

To be carbon neutral, a company must remove the same amount of carbon dioxide that it is emitting into the atmosphere to achieve a net-zero carbon emissions. A carbon negative company, on the other hand, removes more carbon from the atmosphere than it releases.

In view of the significant efforts across the globe of companies to go carbon neutral or be carbon negative, a need exists for methods and processes for producing polymers in a more sustainable way. In this regard, a need exists for a process and method for producing polyoxymethylene polymers that are carbon neutral or carbon negative.

### SUMMARY

In general, the present disclosure is directed to producing polyoxymethylene polymers, particularly polyoxymethylene copolymers, in a way that creates carbon offsets.

In one aspect, the present disclosure is directed to a process for producing a polyoxymethylene polymer. The process includes forming a cyclic acetal from at least one carbon negative component. A comonomer is also formed from at least one carbon negative component. The cyclic acetal and the comonomer are then polymerized in the presence of a catalyst to form a polyoxymethylene copolymer. In accordance with the present disclosure, the carbon negative component can comprise a biogas or a recycled gas that can be collected, for instance, from an industrial process. The biogas, for instance, can be a gas, such as methane, formed from organic waste using anaerobic digestion or gasification technologies. Alternatively, the gas can be a recycled gas, such as carbon dioxide or carbon monoxide, that is collected from an industrial process instead of being released into the environment.

In one aspect, the carbon negative component can be used to produce methanol. Methanol can then be converted into the cyclic acetal and/or the comonomer. The methanol can be produced from biomass or can be produced from a carbon negative gas as described above, such as a biogas or a recycled gas.

In one embodiment, the process further comprises forming a chain transfer agent from at least one carbon negative component and polymerizing the cyclic acetal with the comonomer and the chain transfer agent in the presence of the catalyst to form the polyoxymethylene copolymer. The chain transfer agent, for instance, can comprise methylal. Alternatively, the chain transfer agent can be a glycol. The cyclic acetal, on the other hand, can be trioxane while the comonomer can comprise dioxolane. In one aspect, the polyoxymethylene copolymer contains the comonomer in an amount from about 0.1% to about 5 mol %.

The polyoxymethylene copolymer produced according to the process of the present disclosure can include various different terminal groups. For example, in one embodiment, terminal hydroxyl groups can be present on the polymer in an amount from about 5 mmol/kg to about 150 mmol/kg, such as from about 25 mmol/kg to about 150 mmol/kg.

Other terminal groups that may be present on the polyoxymethylene polymer include alkoxy such as methoxy groups or ethoxy groups. The polymer may also include formate groups and hemiacetal groups.

In one aspect, greater than about 80% by weight, such as greater than about 90% by weight, such as even 100% by weight of carbon contained in the polyoxymethylene copolymer can be derived from one or more carbon negative components.

In another aspect, the present disclosure is directed to a process for producing paraformaldehyde. The process includes forming formaldehyde from at least one carbon negative component. For example, the carbon negative component can comprise a biogas or a recycled gas that can be collected, for instance, from an industrial process. The biogas or recycled gas can be first converted into methanol. The methanol can be then used to produce formaldehyde, which is then polymerized in the presence of a catalyst to form a paraformaldehyde. Paraformaldehyde has the structure of HO(CH2O)ₙH wherein n is from about 8 to about 100. The paraformaldehyde can have a purity of from between 90 and 99% depending on the degree of polymerization n with the remainder being bound or free water. The paraformaldehyde can then be used to synthesize phenols, ureas, furfural alcohols, resorcinol resins, melamine resins, and formaldehyde resins. These products can be used in industrial coatings, wood products, textiles, foundry resins, oil well additives, lubricating oil additives, adhesive resins and electrical component molding materials.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures:

FIG. 1 illustrates one embodiment of a coffee maker apparatus comprising a composition prepared according to the present disclosure;

FIG. 2 illustrates one embodiment of a member of a coffee maker apparatus comprising a composition prepared according to the present disclosure;

FIG. 3 illustrates one embodiment of a medical apparatus comprising a composition prepared according to the present disclosure;

FIG. 4 illustrates another embodiment of a medical apparatus comprising a composition prepared according to the present disclosure;

FIG. 5 illustrates one embodiment of a conveyance apparatus comprising a composition prepared according to the present disclosure; and

FIG. 6 illustrates one embodiment of a cosmetic closure apparatus comprising a composition prepared according to the present disclosure.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to a process for producing polyoxymethylene polymers or paraformaldehyde in a more sustainable manner. In accordance with the present disclosure, polyoxymethylene polymers or paraformaldehyde are formed from carbon negative components. The feedstock used to produce the polyoxymethylene polymers or paraformaldehyde can be a biogas and/or a recycled gas, instead of fossil derived gases, such as natural gas. The biogas or recycled gas, for instance, can be used to produce methanol. The methanol can then be used to produce formaldehyde which can then later be converted into trioxane or other monomers. The methanol can also be used to produce comonomers, chain transfer agents, and the like. These components can then be polymerized together to form a polyoxymethylene polymer that, as described above, is formed from biogases and/or recycled gases. Alternatively, paraformaldehyde can be formed directly from formaldehyde. The formed polyoxymethylene polymer or paraformaldehyde therefore has a much smaller carbon footprint and can even be produced so as to be overall carbon neutral or even carbon negative. Through the use of renewable energy or a mix from different energies with renewable content, the decrease in the carbon footprint can be enhanced even further.

Polyoxymethylene polymers or paraformaldehyde made according to the present disclosure can fulfill the sustainability needs of many manufacturers and consumers. The polyoxymethylene polymer or paraformaldehyde can be used to produce all different types of products and articles in all different fields. The polyoxymethylene polymer, for instance, can be used to produce molded parts and articles for use in the automotive field, electrical field, medical field, and the like. Manufacturers can incorporate the polyoxymethylene polymer into their products in order to meet goals for renewable or bio-based content. Overall, the polyoxymethylene polymer or paraformaldehyde made according to the present disclosure can help manufacturers reduce their carbon footprint without in any way sacrificing quality or mechanical properties.

Ultimately, polyoxymethylene polymers or paraformaldehyde made according to the present disclosure can be certified according to any suitable standard. One such certification is the International Sustainability and Carbon Certification (ISCC). The ISCC is a globally applicable sustainability certification system and covers all sustainable feedstocks, including agricultural and forestry biomass, circular and bio-based materials and renewables. The ISCC follows the mass balance approach in which the renewable content of the polymer can be verified. In mass balance, renewable feedstock is attributed to selected products, according to their individual formulation taking into account all yields and losses. Only raw materials used as feedstock (but not for energy) for the production are considered for mass balancing. The key criteria used for applying the mass balance approach include feedstock qualification, chain of custody, and product claims.

Over the past several years, the mass balance approach has prevailed as the preferred method of validating a sustainable product in the chemical industry as opposed to other methods that may include calculating bio-based content in the actual product. The mass balance approach makes it possible to track the amount and sustainability characteristics of recycled and/or bio-based feedstocks in the value chain and attribute it to the final product in a verifiable manner.

For purely exemplary purposes, for instance, under the mass balance approach, forming methanol from fossil fuels, such as natural gas, creates a greenhouse gas footprint and is carbon positive. The amount of carbon released into the environment can be calculated by taking into account production, combustion and transport. If methanol is not combusted, emissions for combustion are not taken into account resulting in a carbon footprint of about 16 g CO₂ eq/MJ for production and approximately 1.4 g CO₂ eq/MJ for transport. If, on the other hand, the methanol is produced from a biogas or a recycled gas (such as a recycled gas from an industrial process), the use of the methanol for chemical applications can render the final product carbon neutral or even carbon negative. The use of a biogas or of a recycled gas for the production of methanol, for instance, can be considered a carbon sink. The carbon sink can be calculated to be approximately 40 g CO₂ eq/MJ. The carbon sink calculation can then be subtracted from the production and transport carbon footprint calculated above. Consequently, according to this exemplary calculation, using methanol derived from a biogas or a recycled gas results in a negative carbon footprint of from about −30 g CO₂ eq/MJ to about −45 g CO₂ eq/MJ and a carbon sink that can possibly be as high as −0.860 KG CO₂/kg.

In order to produce polyoxymethylene polymers in accordance with the present disclosure, methanol is first formed from a biogas or from a recycled gas. Methanol, for instance, is the primary raw material in the production of polyoxymethylene polymers. In the past, methanol was produced from fossilized natural gas which forms what is known in the art as “grey” methanol. In general, approximately 1.1 kg to about 1.3 kg of methanol is needed to produce 1 kg of a polyoxymethylene polymer. The methanol is first used to form formaldehyde, which is then used to form a cyclic acetal such as trioxane, and which is then polymerized to form the polyoxymethylene polymer. When producing polyoxymethylene copolymers, the polyoxymethylene polymer can contain a comonomer generally in an amount from about 0.1 mol % to about 5 mol %. In one aspect, the comonomer is 1,3-dioxolane. The comonomer can also be produced from methanol via formaldehyde and ethylene glycol.

In accordance with the present disclosure, the methanol is produced from a biogas or from a recycled gas. In one aspect, the biogas is methane produced from solid waste landfills and anaerobic digestion plants. It was discovered that the use of a biogas is much more efficient than other bio sources, such as glycerine.

Conversion of a methane biogas to methanol can be carried out using different processes and steps. In one embodiment, for instance, methane can be directly converted into methanol via the partial oxidation of methane in the presence of a metal-containing zeolite catalyst. In this embodiment, one mol of methane is reacted with 0.5 mols of molecular oxygen to yield methanol.

In an alternative embodiment, the biogas methane can be converted into a syngas, which is produced by steam reforming the methane. The syngas, for instance, can contain carbon monoxide or carbon dioxide. Methanol can then be produced from the carbon monoxide or the carbon dioxide using the following reaction schemes:

CO+2H₂→CH₃OH

CO₂+3H₂→CH₃OH+H₂O

The above reactions can be carried out using a copper-based catalyst.

In addition to using a methane biogas, the methanol can also be produced using recycled gases according to the present disclosure. The recycled gases, for instance, can be obtained from an industrial process. The recycled gases, for instance, represent gases that would normally be released to the atmosphere and contain carbon. By collecting these gases and using them to produce methanol, the carbon footprint of the resulting polymer is greatly reduced.

The recycled gas, for instance, can comprise carbon dioxide, carbon monoxide, or a combination of carbon dioxide and carbon monoxide. The above reaction schemes can then be used to convert the recycled gas into methanol.

Through the process described above, a methanol product is produced that is formed completely from sustainable resources. In order to produce a polyoxymethylene polymer, the methanol can be converted into formaldehyde and then converted into a cyclic acetal monomer, such as trioxane. The methanol can also be used to produce comonomers and chain transfer agents.

The preparation of formaldehyde from methanol can be carried out also using various different processes and techniques. In one embodiment, for instance, the formation of formaldehyde is carried out through oxidation using the following reaction scheme:

CH₃OH+1/2O₂→CH₂O+H₂O

over catalysts comprising iron oxide and molybdenum oxide at from 300° C. to 450° C. In another embodiment, oxidative dehydrogenation is used to produce methanol according to the following reaction scheme:

CH₃OH→CH₂O+H₂

H₂+1/2O₂→H₂O

at from 600° C. to 720° C.

In still another embodiment, formaldehyde can be produced from methanol using dehydrogenation in a non-oxidative process according to the following equation:

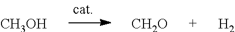

Suitable catalysts are known, for example, from the literature, see, for example, Chem. Eng. Technol. 1994, 17, 34.

Suitable metals are, for example, Li, Na, K, Cs, Mg, Al, In, Ga, Ag, Cu, Zn, Fe, Ni, Co, Mo, Ti, Pt or their compounds. Also suitable are, for example, S, Se, phosphates of transition metals such as V and Fe, and heteropolyacids such as molybdophosphoric acid.

Examples of specific catalysts are:
sodium or sodium compounds (DE-A-37 19 055 and DE-A-38 11 509) aluminum oxide, alkali metal aluminate and/or alkaline earth metal aluminate (EP-A04 05 348)
silver oxide (JP-A 60/089 441, Derwent Report 85-15 68 91/26)
a catalyst comprising copper, zinc and sulfur (DE-A 25 25 174)
a catalyst comprising copper, zinc and selenium (U.S. Pat. No. 4,054,609)
a catalyst comprising zinc and/or indium (EP-A 0 130 068)
silver (U.S. Pat. No. 2,953,602)
silver, copper and silicon (U.S. Pat. No. 2,939,883)
compounds containing zinc, cadmium, selenium, tellurium or indium.

Particular preference is given to sodium compounds selected from the group consisting of:
a) sodium alkoxides,
b) sodium carboxylates,
c) sodium salts of C—H acid compounds,
d) sodium oxide, sodium hydroxide, sodium nitrite, sodium acetylide, sodium carbide, sodium hydride and sodium carbonyl.

Once formaldehyde is produced from the methanol, the formaldehyde can then be used to produce monomers, comonomers, and any other components used during the polymerization of the polyoxymethylene polymer. In one embodiment, the formaldehyde is used to produce a cyclic acetal as the primary monomer for producing the polyoxymethylene polymer. In order to produce a cyclic acetal, the formaldehyde source reacts or converts in the presence of a catalyst. The catalyst can be a cationic catalyst, such as Bronsted acids or Lewis acids.

The catalyst is a catalyst for the conversion (reaction) of a formaldehyde source into cyclic acetals, in particular into trioxane and/or tetroxane.

Cyclic acetals relate to cyclic acetals derived from formaldehyde. Typical representatives are showing the following formula:

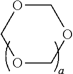

wherein a is an integer ranging from 1 to 3.

Preferably, the cyclic acetals produced by the process are trioxane (a=1) and/or tetroxane (a=2). Trioxane and Tetroxane usually form the major part (at least 80 wt.-%, preferably at least 90 wt.-%) of the cyclic acetals formed by the process.

The weight ratio of trioxane to tetroxane varies with the catalyst used. Typically, the weight ratio of trioxane to tetroxane ranges from about 3:1 to about 40:1, preferably about 4:1 to about 20:1.

In one embodiment, the above reaction can occur in the presence of an aprotic compound as described in U.S. Pat. No. 9,604,956, which is incorporated herein by reference. The aprotic compound, for instance, can be a cyclic or alicyclic organic sulfoxide, an alicyclic or cyclic sulfone, or the like. In one embodiment, the aprotic compound is sulfolane. The presence of the aprotic compound can greatly enhance conversion rates.

In addition to producing the primary monomer, the methanol of the present disclosure derived from a biogas or a recycled gas can also be used to produce one or more comonomers. The one or more comonomers may include cyclic ethers or acetals. In one embodiment, the comonomer is a dioxolane, such as 1,3-dioxolane. Of particular advantage, the comonomer can also be produced from the methanol of the present disclosure produced from a biogas or a recycled gas. In one embodiment, the comonomer can be produced via formaldehyde that is obtained or derived from the methanol. In addition to formaldehyde, a glycol, such as ethylene glycol can also be used to produce the comonomer. The ethylene glycol can be produced from one or more carbon negative components.

During the polymerization of polyoxymethylene polymers, chain transfer agents are also commonly used in order to control molecular weight and/or control the formation of end groups. Of particular advantage, the chain transfer agents used in the process can also be formed from the biogas or recycled gas via methanol. For example, one chain transfer agent, methylal, can be formed by the oxidation of methanol or by the reaction of formaldehyde with methanol.

Once the building blocks of the polymer are produced from the biogas and/or recycled gas, the different components can then be used to produce the polyoxymethylene polymer.

The preparation of the polyoxymethylene polymer can be carried out by polymerization of polyoxymethylene-forming monomers, such as trioxane or a mixture of trioxane and a cyclic acetal such as dioxolane in the presence of a molecular weight regulator, such as a glycol or methylal. The polyoxymethylene polymer used in the polymer composition may comprise a homopolymer or a copolymer. Although homopolymers can be produced, the present disclosure is primarily directed to producing polyoxymethylene copolymers which comprise at least 50 mol. %, such as at least 75 mol. %, such as at least 90 mol. % and such as even at least 97 mol. % of —CH₂O-repeat units.

In one embodiment, a polyoxymethylene copolymer is used. The copolymer can contain from about 0.01 mol. % to about 20 mol. % and in particular from about 0.5 mol. % to about 10 mol. % of repeat units that comprise a saturated or ethylenically unsaturated alkylene group having at least 2 carbon atoms, or a cycloalkylene group, which has sulfur atoms or oxygen atoms in the chain and may include one or more substituents selected from the group consisting of alkyl cycloalkyl, aryl, aralkyl, heteroaryl, halogen or alkoxy. In one embodiment, a cyclic ether or acetal is used that can be introduced into the copolymer via a ring-opening reaction.

Preferred cyclic ethers or acetals are those of the formula:

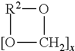

in which x is 0 or 1 and R² is a C₂-C₄-alkylene group which, if appropriate, has one or more substituents which are C₁-C₄-alkyl groups, or are C₁-C₄-alkoxy groups, and/or are halogen atoms, preferably chlorine atoms. Merely by way of example, mention may be made of ethylene oxide, propylene 1,2-oxide, butylene 1,2-oxide, butylene 1,3-oxide, 1,3-dioxane, 1,3-dioxolane, and 1,3-dioxepan as cyclic ethers, and also of linear oligo- or polyformals, such as polydioxolane or polydioxepan, as comonomers. It is particularly advantageous to use copolymers composed of from 99.5 to 95 mol. % of trioxane and of from 0.01 to 5 mol. %, such as from 0.5 to 4 mol. %, of one of the above-mentioned comonomers. In one embodiment, the polyoxymethylene polymer contains relatively low amounts of comonomer. For instance, the comonomer can be present in an amount less than about 2 mol. %, such as less than about 1.5 mol. %, such as less than about 1 mol. %, such as less than about 0.8 mol. %, such as less than about 0.6 mol. %.

The polymerization can be effected as precipitation polymerization or in the melt. By a suitable choice of the polymerization parameters, such as duration of polymerization or amount of molecular weight regulator, the molecular weight and hence the MVR value of the resulting polymer can be adjusted.

In one embodiment, the polyoxymethylene polymer used in the polymer composition may contain a relatively high amount of reactive groups or functional groups in the terminal positions. The reactive groups, for instance, may comprise —OH or —NH₂ groups.

In one embodiment, the polyoxymethylene polymer can have terminal hydroxyl groups, for example hydroxyethyl endgroups and/or hemiformal endgroups in at least more than about 50% of all the terminal sites on the polymer. For instance, the polyoxymethylene polymer may have at least about 70%, such as at least about 80%, such as at least about 85% of its terminal groups be hydroxyl groups, based on the total number of terminal groups present. It should be understood that the total number of terminal groups present includes all end and side terminal groups.

In one embodiment, the polyoxymethylene polymer has a content of terminal hydroxyl groups of at least 15 mmol/kg, such as at least 18 mmol/kg, such as at least 20 mmol/kg. In one embodiment, the terminal hydroxyl group content ranges from 18 to 50 mmol/kg. In an alternative embodiment, the polyoxymethylene polymer may contain terminal hydroxyl groups in an amount less than 20 mmol/kg, such as less than 18 mmol/kg, such as less than 15 mmol/kg. For instance, the polyoxymethylene polymer may contain terminal hydroxyl groups in an amount from about 5 mmol/kg to about 20 mmol/kg, such as from about 5 mmol/kg to about 15 mmol/kg. For example, a polyoxymethylene polymer may be formed that has a lower terminal hydroxyl group content.

In addition to or instead of the terminal hydroxyl groups, the polyoxymethylene polymer may also have other terminal groups. Examples of these are alkoxy groups, formate groups, acetate groups or aldehyde groups. According to one embodiment, the polyoxymethylene is a homo- or copolymer which comprises at least 50 mol-%, such as at least 75 mol-%, such as at least 90 mol-% and such as even at least 95 mol-% of —CH₂O-repeat units.

In one embodiment, a polyoxymethylene polymer can be produced using a cationic polymerization process followed by solution hydrolysis to remove any unstable end groups. During cationic polymerization, a glycol, such as ethylene glycol or methylal can be used as a chain terminating agent. A heteropoly acid, triflic acid or a boron compound may be used as the catalyst.

The polyoxymethylene polymer can have any suitable molecular weight. The molecular weight of the polymer, for instance, can be from about 4,000 grams per mole to about 20,000 g/mol. In other embodiments, however, the molecular weight can be well above 20,000 g/mol, such as from about 20,000 g/mol to about 100,000 g/mol.

The polyoxymethylene polymer present in the composition can generally have a melt flow index (MFI) ranging from about 0.1 to about 120 cm³/10 min, as determined according to ISO 1133 at 190° C. and 2.16 kg. In one embodiment, the polyoxymethylene polymer may have a melt flow index of greater than about 1 cm³/10 min, such as greater than about 2 cm³/10 min, such as greater than about 5 cm³/10 min, such as greater than about 10 cm³/10 min, such as greater than about 20 cm³/10 min, such as greater than about 30 cm³/10 min. The polymer may, in some cases, have a melt flow index of less than about 55 cm³/10 min, such as less than about 45 cm³/10 min, such as less than about 35 cm³/10 min, such as less than about 25 cm³/10 min, such as less than about 15 cm³/10 min, such as less than about 10 cm³/10 min, such as less than about 5 cm³/10 min.

In addition to polyoxymethylene polymers, the present disclosure is also directed to a process for producing paraformaldehyde. The process includes forming formaldehyde from at least one carbon negative component. For example, the carbon negative component can comprise a biogas or a recycled gas that can be collected, for instance, from an industrial process. The biogas or recycled gas can be first converted into methanol. The methanol can be then used to produce formaldehyde as described above, which is then polymerized in the presence of a catalyst to form a paraformaldehyde. Paraformaldehyde has the structure of HO(CH2O)ₙH wherein n is from about 8 to about 100. The paraformaldehyde can have a purity of from between 90 and 99% depending on the degree of polymerization n with the remainder being bound or free water. The paraformaldehyde can then be used to synthesize phenols, ureas, furfural alcohols, resorcinol resins, melamine resins, and formaldehyde resins. These products can be used in industrial coatings, wood products, textiles, foundry resins, oil well additives, lubricating oil additives, adhesive resins and electrical component molding materials.

The polyoxymethylene polymer of the present disclosure can be used in neat form or can be combined with various additives and components. For instance, in one embodiment, the polyoxymethylene polymer can be combined with a tribological modifier.

For example, ultra-high molecular weight silicone (UHMW-Si) may be used to modify the polyoxymethylene polymer. In general, the UHMW-Si can have an average molecular weight of greater than 100,000 g/mol, such as greater than about 200,000 g/mol, such as greater than about 300,000 g/mol, such as greater than about 500,000 g/mol and less than about 3,000,000 g/mol, such as less than about 2,000,000 g/mol, such as less than about 1,000,000 g/mol, such as less than about 500,000 g/mol, such as less than about 300,000 g/mol. Generally, the UHMW-Si can have a kinematic viscosity at 40° C. measured according to DIN 51562 of greater than 100,000 mm² s⁻¹, such as greater than about 200,000 mm² s⁻¹, such as greater than about 1,000,000 mm² s⁻¹, such as greater than about 5,000,000 mm² s⁻¹, such as greater than about 10,000,000 mm² s⁻¹, such as greater than about 15,000,000 mm² s⁻¹ and less than about 50,000,000 mm² s⁻¹, such as less than about 25,000,000 mm² s⁻¹, such as less than about 10,000,000 mm² s⁻¹, such as less than about 1,000,000 mm² s⁻¹, such as less than about 500,000 mm² s⁻¹, such as less than about 200,000 mm² s⁻¹.

In still another embodiment, the tribological modifier may comprise a polytetrafluoroethylene. The polytetrafluoroethylene may be in the form of a powder and can be present in the polymer composition in an amount from about 1% to about 10% by weight.

According to the present disclosure, various other tribological modifiers may be incorporated into the polyoxymethylene polymer composition. These tribological modifiers may include, for instance, calcium carbonate particles, ultrahigh-molecular-weight polyethylene (UHMW-PE) particles, stearyl stearate particles, silicone oil, a polyethylene wax, an amide wax, wax particles comprising an aliphatic ester wax comprised of a fatty acid and a monohydric alcohol, a graft copolymer with an olefin polymer as a graft base, or a combination thereof. These tribological modifiers include the following:

(1) From 0.1-50 wt. %, such as from 1-25 wt. %, of a calcium carbonate particle such as a calcium carbonate (chalk) powder.

(2) From 0.1-50 wt. %, such as from 1-25 wt. %, such as from 2.5-20 wt. %, such as from 5 to 15 wt. %, of an ultrahigh-molecular-weight polyethylene (UHMW-PE) powder. UHMW-PE can be employed as a powder, in particular as a micro-powder. The UHMW-PE generally has a mean particle diameter D₅₀ (volume based and determined by light scattering) in the range of 1 to 5000 μm, preferably from 10 to 500 μm, and particularly preferably from 10 to 150 μm such as from 30 to 130 μm, such as from 80 to 150 μm, such as from 30 to 90 μm.

The UHMW-PE can have an average molecular weight of higher than 1.0·10⁶ g/mol, such as higher than 2.0·10⁶ g/mol, such as higher than 4.0·10⁶ g/mol, such as ranging from 1.0·10⁶ g/mol to 15.0·10⁶ g/mol, such as from 3.0·10⁶ g/mol to 12.0·10⁶ g/mol, determined by viscosimetry. Preferably, the viscosity number of the UHMW-PE is higher than 1000 ml/g, such as higher than 1500 ml/g, such as ranging from 1800 ml/g to 5000 ml/g, such as ranging from 2000 ml/g to 4300 ml/g (determined according to ISO 1628, part 3; concentration in decahydronaphthalin: 0.0002 g/ml).

(3) From 0.1-10 wt. %, such as from 0.1-5 wt. %, such as from 0.5-3 wt. %, of stearyl stearate.

(4) From 0.1-10 wt. %, such as from 0.5-5 wt. %, such as from 0.8-2 wt. %, of a silicone oil. Alternatively, in one embodiment, the composition may be substantially free of silicone oil, such that the silicone oil is present in an amount of less than about 0.1 wt. %, such as less than about 0.05 wt. %, such as less than about 0.01 wt. %, such as about 0 wt. %.

(5) From 0.1-5 wt. %, such as from 0.5-3 wt. %, of a polyethylene wax, such as an oxidized polyethylene wax.

(6) From 0.1-5 wt. %, such as from 0.2-2 wt. %, of an amide wax.

(7) From 0.1-5 wt. %, such as from 0.5-3 wt. %, of an aliphatic ester wax composed of a fatty acid and of a monohydric alcohol.

The polymer composition of the present disclosure may also contain other known additives such as, for example, antioxidants, formaldehyde scavengers, acid scavengers, UV stabilizers or heat stabilizers, reinforcing fibers. In addition, the compositions can contain processing auxiliaries, for example adhesion promoters, lubricants, nucleants, demolding agents, fillers, or antistatic agents and additives which impart a desired property to the compositions and articles or parts produced therefrom.

In one embodiment, an ultraviolet light stabilizer may be present. The ultraviolet light stabilizer may comprise a benzophenone, a benzotriazole, or a benzoate. The UV light absorber, when present, may be present in the polymer composition in an amount of at least about 0.01 wt. %, such as at least about 0.05 wt. %, such as at least about 0.075 wt. % and less than about 1 wt. %, such as less than about 0.75 wt. %, such as less than about 0.5 wt. %, wherein the weight is based on the total weight of the respective polymer composition.

In one embodiment, a formaldehyde scavenger, such as a nitrogen-containing compound, may be present. Mainly, of these are heterocyclic compounds having at least one nitrogen atom as hetero atom which is either adjacent to an amino-substituted carbon atom or to a carbonyl group, for example pyridine, pyrimidine, pyrazine, pyrrolidone, aminopyridine and compounds derived therefrom. Other particularly advantageous compounds are triamino-1,3,5-triazine (melamine) and its derivatives, such as melamine-formaldehyde condensates and methylol melamine. Oligomeric polyamides are also suitable in principle for use as formaldehyde scavengers. The formaldehyde scavenger may be used individually or in combination.

Further, the formaldehyde scavenger may be a guanamine compound which may include an aliphatic guanamine-based compound, an alicyclic guanamine-based compound, an aromatic guanamine-based compound, a hetero atom-containing guanamine-based compound, or the like. The formaldehyde scavenger may be present in the polymer composition in an amount of at least about 0.01 wt. %, such as at least about 0.05 wt. %, such as at least about 0.075 wt. % and less than about 1 wt. %, such as less than about 0.75 wt. %, such as less than about 0.5 wt. %, wherein the weight is based on the total weight of the respective polymer composition.

In one embodiment, an acid scavenger may be present. The acid scavenger may comprise, for instance, an alkaline earth metal salt. For instance, the acid scavenger may comprise a calcium salt, such as a calcium citrate. The acid scavenger may be present in an amount of at least about 0.001 wt. %, such as at least about 0.005 wt. %, such as at least about 0.0075 wt. % and less than about 1 wt. %, such as less than about 0.75 wt. %, such as less than about 0.5 wt. %, wherein the weight is based on the total weight of the respective polymer composition.

In one embodiment, a nucleant may be present. The nucleant may increase crystallinity and may comprise an oxymethylene terpolymer. In one particular embodiment, for instance, the nucleant may comprise a terpolymer of butanediol diglycidyl ether, ethylene oxide, and trioxane. The nucleant may be present in the composition in an amount of at least about 0.01 wt. %, such as at least about 0.05 wt. %, such as at least about 0.1 wt. % and less than about 2 wt. %, such as less than about 1.5 wt. %, such as less than about 1 wt. %, wherein the weight is based on the total weight of the respective polymer composition.

In one embodiment, an antioxidant, such as a sterically hindered phenol, may be present. Examples which are available commercially, are pentaerythrityl tetrakis[3-(3,5-di-tert-butyl-4-hydroxyphenyl)propionate], triethylene glycol bis[3-(3-tert-butyl-4-hydroxy-5-methylphenyl)propionate], 3,3′-bis[3-(3,5-di-tert-butyl-4-hydroxyphenyl)propionohydrazide], and hexamethylene glycol bis[3-(3,5-di-tert-butyl-4-hydroxyphenyl)propionate]. The antioxidant may be present in the polymer composition in an amount of at least about 0.01 wt. %, such as at least about 0.05 wt. %, such as at least about 0.075 wt. % and less than about 1 wt. %, such as less than about 0.75 wt. %, such as less than about 0.5 wt. %, wherein the weight is based on the total weight of the respective polymer composition.

In one embodiment, a light stabilizer, such as a sterically hindered amine, may be present in addition to the ultraviolet light stabilizer. Hindered amine light stabilizers that may be used include oligomeric hindered amine compounds that are N-methylated. For instance, hindered amine light stabilizer may comprise a high molecular weight hindered amine stabilizer. The light stabilizers, when present, may be present in the polymer composition in an amount of at least about 0.01 wt. %, such as at least about 0.05 wt. %, such as at least about 0.075 wt. % and less than about 1 wt. %, such as less than about 0.75 wt. %, such as less than about 0.5 wt. %, wherein the weight is based on the total weight of the respective polymer composition.

In one embodiment, a lubricant, not including the tribological modifiers mentioned above, may be present. The lubricant may comprise a polymer wax composition. Further, in one embodiment, a polyethylene glycol polymer (processing aid) may be present in the composition. The polyethylene glycol, for instance, may have a molecular weight of from about 1000 to about 5000, such as from about 3000 to about 4000. In one embodiment, for instance, PEG-75 may be present. In another embodiment, a fatty acid amide such as ethylene bis(stearamide) may be present. Lubricants may generally be present in the polymer composition in an amount of at least about 0.01 wt. %, such as at least about 0.05 wt. %, such as at least about 0.075 wt. % and less than about 1 wt. %, such as less than about 0.75 wt. %, such as less than about 0.5 wt. %, wherein the weight is based on the total weight of the respective polymer composition.

In one embodiment, a colorant may be present. Colorants that may be used include any desired inorganic pigments, such as titanium dioxide, ultramarine blue, cobalt blue, and other organic pigments and dyes, such as phthalocyanines, anthraquinnones, and the like. Other colorants include carbon black or various other polymer-soluble dyes. The colorant may be present in the composition in an amount of at least about 0.01 wt. %, such as at least about 0.05 wt. %, such as at least about 0.1 wt. % and less than about 5 wt. %, such as less than about 2.5 wt. %, such as less than about 1 wt. %, wherein the weight is based on the total weight of the respective polymer composition.

In one embodiment, a reinforcing fiber may be present. The reinforcing fibers which may be used according to the present invention include mineral fibers, glass fibers, polymer fibers such as aramid fibers, metal fibers such as steel fibers, carbon fibers, or natural fibers. These fibers may be unmodified or modified, e.g. provided with a sizing or chemically treated, in order to improve adhesion to the polymer. Fiber diameters can vary depending upon the particular fiber used and whether the fiber is in either a chopped or a continuous form. The fibers, for instance, can have a diameter of from about 5 μm to about 100 μm, such as from about 5 μm to about 50 μm, such as from about 5 μm to about 15 μm. When present, the respective composition may contain reinforcing fibers in an amount of at least 1 wt. %, such as at least 5 wt. %, such as at least 7 wt. %, such as at least 10 wt. %, such as at least 15 wt. % and generally less than about 50 wt. %, such as less than about 45 wt. %, such as less than about 40 wt. %, such as less than about 30 wt. %, such as less than about 20 wt. %, wherein the weight is based on the total weight of the respective polyoxymethylene polymer composition.

The polymer composition may also comprise an impact modifier such as a thermoplastic elastomer. Thermoplastic elastomers are materials with both thermoplastic and elastomeric properties. Thermoplastic elastomers include styrenic block copolymers, polyolefin blends referred to as thermoplastic olefin elastomers, elastomeric alloys, thermoplastic polyurethanes, thermoplastic copolyesters, and thermoplastic polyamides.

Thermoplastic elastomers well suited for use in the present disclosure are polyester elastomers (TPE-E), thermoplastic polyamide elastomers (TPE-A) and in particular thermoplastic polyurethane elastomers (TPE-U).

Alternatively, the impact modifier can be core-shell type impact modifier. For example, the impact modifier can comprise metacrylate-butadiene-styrene copolymer particles.

The amount of thermoplastic elastomer contained in the polymer composition can vary depending upon various factors. For instance, the thermoplastic elastomer can be present in an amount ranging from about 0.5% by weight to about 50% by weight. In one embodiment, for instance, a thermoplastic elastomer or impact modifier may be present in the composition in an amount less than about 25% by weight, such as in an amount less than about 15% by weight, such as in an amount less than about 10% by weight. The thermoplastic elastomer or impact modifier is generally present in an amount greater than about 2% by weight, such as in an amount greater than about 5% by weight, such as in an amount greater than about 8% by weight, such as in an amount greater than about 10% by weight.

The polymer composition of the present disclosure can be used to produce various molded parts. The parts can be formed through any suitable molding process, such as an injection molding process or through a blow molding process. Polymer articles that may be made in accordance with the present disclosure include knobs, door handles, automotive decorative trim pieces, and the like without limitation. Other polymer articles, for instance, that may be made in accordance with the present disclosure include latches, levers, gears, pivot housings, speaker grills, and the like.

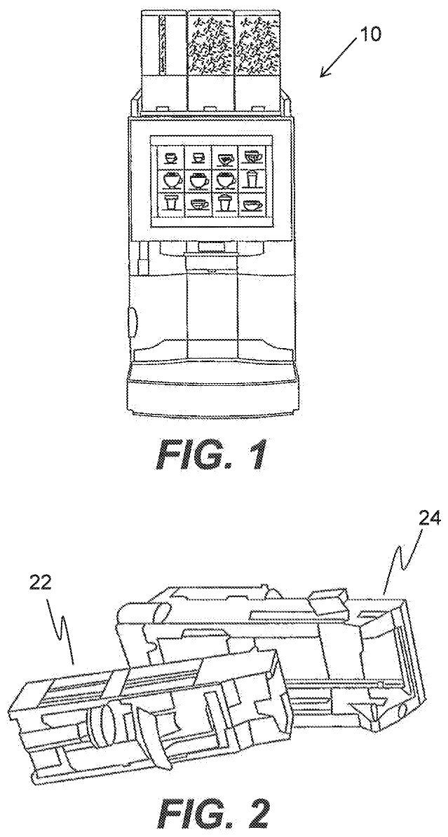

In one particular embodiment, the polymer composition is used to make a coffeemaker 10 as shown in FIG. 1. Coffeemakers are designed to heat liquids very rapidly and produce a heated beverage. Consequently, the operating environment, especially the internal operating environment, of a coffeemaker can change from room temperature to high temperature relatively quickly. Coffeemakers, such as coffeemaker 10 as shown in FIG. 1, also have many parts. In accordance with the present disclosure, various internal and external parts of the coffeemaker can be made from the polymer composition of the present disclosure, especially parts that are exposed to high temperature water.

A coffee making apparatus typically comprises a water heating unit, a coffee supply unit, and a brewing assembly. For the production of a heated beverage, coffee is fed from the coffee supply unit and heated water from the water heating unit to the brewing assembly. Typical brewing assemblies comprise a brewing head, an upper closure element, a lower closure element, and at least one linear guide element.

Referring to FIG. 2, the coffeemaker 10 can include a first member 22 and a second member 24. The first member 22 and the second member 24, in one embodiment, can be part of the brewing assembly or part of the coffee supply unit. For example, the members 22 and 24 can be for receiving and loading coffee into a designated area, such as a capsule, for producing a coffee beverage. Because of close proximity to heated water, the members 22 and 24 may experience an operating environment well above 30° C., such as greater than 40° C., such as greater than 50° C., such as even greater than 60° C.

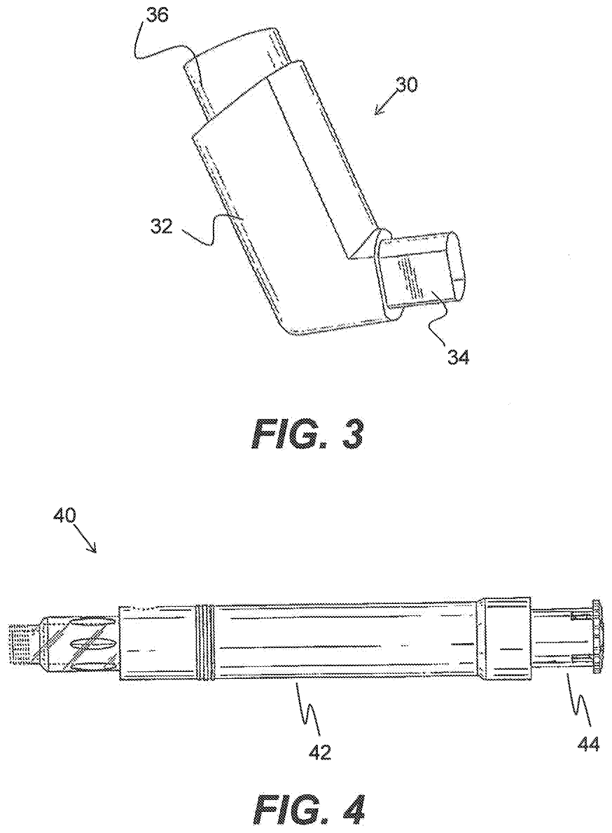

Referring to FIG. 3, an inhaler 30 is shown that may be made from the polyoxymethylene polymer. The inhaler 30 includes a housing 32 attached to a mouthpiece 34. In operative association with the housing 32 is a plunger 36 for receiving a canister containing a composition to be inhaled. The composition may comprise a spray or a powder.

During use, the inhaler 30 administers metered doses of a medication, such as an asthma medication to a patient. The asthma medication may be suspended or dissolved in a propellant or may be contained in a powder. When a patient actuates the inhaler to breathe in the medication, a valve opens allowing the medication to exit the mouthpiece. In accordance with the present disclosure, the housing 32, the mouthpiece 34 and the plunger 36 can all be made from a polymer composition as described above.

Referring to FIG. 4, another medical product that may be made in accordance with the present disclosure is shown. In FIG. 4, a medical injector 40 is illustrated. The medical injector 40 includes a housing 42 in operative association with a plunger 44. The housing 42 may slide relative to the plunger 44. The medical injector 40 may be spring loaded. The medical injector is for injecting a drug into a patient typically into the thigh or the buttocks. The medical injector can be needleless or may contain a needle. When containing a needle, the needle tip is typically shielded within the housing prior to injection. Needleless injectors, on the other hand, can contain a cylinder of pressurized gas that propels a medication through the skin without the use of a needle. In accordance with the present disclosure, the housing 42 and/or the plunger 44 can be made from a polymer composition as described above.

In one embodiment, polymer articles made in accordance with the present disclosure can be used to make components of a conveyor system. Conveyor systems, for instance, typically include a conveyor chain that moves over a track. Such conveyor systems can be used to move all different types of products and goods. In one embodiment, for instance, such conveyors are used to transport food products.

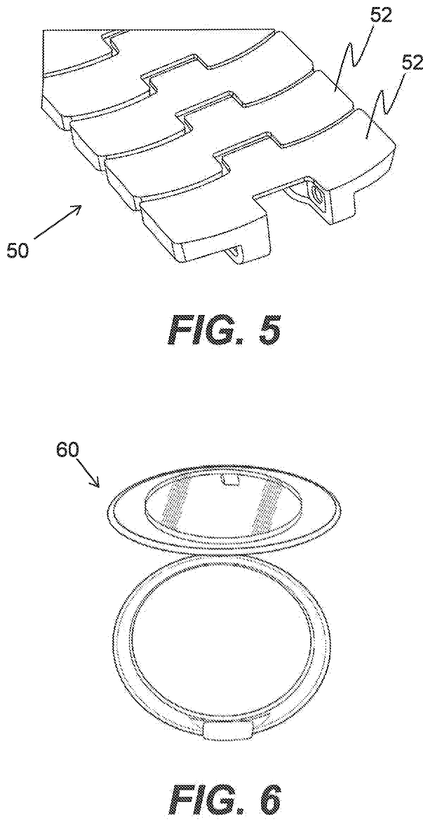

Referring to FIG. 5, for instance, one embodiment of a portion of a conveyor chain 50 is illustrated. As shown, the conveyor chain 50 is made from a plurality of conveyor components 52 or links. Each of the conveyor components 52 includes a top surface for receiving and transporting food products. In accordance with the present disclosure, the conveyor component 52 can be made from the polymer composition of the present disclosure. Of particular advantage, the conveyor component 52 can include one or more coloring agents that provide the components with a desired surface appearance. Advantageously, components comprising compositions prepared according to the present disclosure may exhibit low formaldehyde emission and extraction when exposed to harsh cleaning conditions.

In yet another embodiment of the present disclosure, the polymer composition can be coated with a metal to produce cosmetic closures. For instance, FIG. 6 illustrates one embodiment of a cosmetic closure 60 that may be made in accordance with the present disclosure.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A process for producing a polyoxymethylene polymer comprising:
forming a cyclic acetal from at least one carbon negative component comprising a biogas or a recycled gas;
forming a comonomer from at least one carbon negative component; and
polymerizing the cyclic acetal with the comonomer in the presence of a catalyst to form a polyoxymethylene copolymer.

2. A process as defined in claim 1, wherein the process further comprises forming a chain transfer agent from at least one carbon negative component and polymerizing the cyclic acetal with the comonomer and the chain transfer agent in the presence of the catalyst to form the polyoxymethylene copolymer.

3. A process as defined in claim 2, wherein the at least one carbon negative component is used to form aqueous formaldehyde solutions for producing the cyclic acetal, the comonomer and the chain transfer agent.

4. A process as defined in claim 1, wherein the carbon negative component is used to first form methanol.

5. A process as defined in claim 1, wherein the comonomer comprises dioxolane and wherein the polyoxymethylene copolymer contains 0.5 to 5 mol % comonomer.

6. A process as defined in claim 2, wherein the chain transfer agent comprises methylal or a glycol.

7. A process as defined in claim 1, wherein the chain transfer agent is used in an amount of 100 ppm to 1500 ppm, based on the weight of the polyoxymethylene polymer.

8. A process as defined in claim 1, wherein the polyoxymethylene copolymer includes terminal hydroxyl groups and has a content of terminal hydroxyl groups of from about 5 mmol/kg to about 150 mmol/kg.

9. A process as defined in claim 8, wherein the polyoxymethylene polymer further includes terminal groups comprised of alkoxy groups.

10. A process as defined in claim 1, wherein the polyoxymethylene copolymer includes terminal groups, the terminal groups comprising methoxy groups, ethoxy groups, formate groups and hemiacetal groups.

11. A process as defined in claim 1, wherein greater than about 80% by weight of carbon contained in the polyoxymethylene copolymer is derived from the carbon negative component.

12. A process for producing a polyoxymethylene polymer comprising:
collecting a carbon dioxide byproduct from an industrial process;
combining the carbon dioxide with a hydrogen source to form methanol;
forming a formaldehyde solution;
forming a cyclic acetal from the formaldehyde solution; and
polymerizing the cyclic acetal in the presence of a catalyst to form a polyoxymethylene polymer.

13. A process as defined in claim 12, further comprising forming a comonomer from the carbon dioxide; and
polymerizing the cyclic acetal with the comonomer in the presence of a chain transfer agent and a catalyst to form the polyoxymethylene polymer, the polyoxymethylene polymer comprising a polyoxymethylene copolymer.

14. A process as defined in claim 12, wherein the cyclic acetal comprises trioxane, wherein the comonomer comprises dioxolane; and wherein the polyoxymethylene copolymer contains 0.5 to 5 mol % comonomer.

15. A process as defined in claim 14, wherein the comonomer comprises 1,3-dioxolane.

16. A process as defined in claim 12, wherein the polyoxymethylene copolymer includes terminal hydroxyl groups and has a content of terminal hydroxyl groups of from about 25 mmol/kg to about 150 mmol/kg.

17. A process as defined in claim 16, wherein the polyoxymethylene polymer further includes terminal groups comprised of alkoxy groups.

18. A process as defined in claim 12, wherein greater than about 50% by weight of carbon contained in the polyoxymethylene copolymer is derived from the carbon dioxide.

19. A process for producing paraformaldehyde comprising:
forming formaldehyde from at least one carbon negative component comprising a biogas or a recycled gas; and
polymerizing the formaldehyde in the presence of a catalyst to form paraformaldehyde.

20. A process as defined in claim 19, wherein the carbon negative component is used to first form methanol.
